(19) United States
(12) Patent Application Publication
(10) Pub. No.: US 2010/0021980 A1
(43) Pub. Date: Jan. 28, 2010

(54) HYBRID PROCESS FOR THE PRODUCTION OF BIOFUEL

(75) Inventors: Robert Victor McDonald, Superior, CO (US); Ruben Dario Rodriguez Quintero, Aberdeen (GB); Athanasios Sotiriadis, Bicester (GB)

Correspondence Address: KEAN, MILLER, HAWTHORNE, D'ARMOND,;MCCOWAN & JARMAN, L.L.P., ONE AMERICAN PLACE, 22ND FLOOR, P.O. BOX 3513, BATON ROUGE, LA, 70821, US

(73) Assignee: Shaw Intellectual Property Holdings, Inc., Baton Rouge, LA (US)

(21) Appl. No.: 12/507,884

(22) Filed: Jul. 23, 2009

Related U.S. Application Data
(60) Provisional application No. 61/082,958, filed on Jul. 23, 2008.

(51) Int. Cl. C12P 7/06

(52) U.S. Cl. 435/161

(57) ABSTRACT

A method of producing a biofuel from biomass is provided. In one embodiment, the biofuel production process is an ethanol production process. Various embodiments of the invention include features such as application of a shock wave to biomass, variations in composition of biomass feed stock, continuous fermentation, and anaerobic digestion of stillage to produce a methane based fuel.

### CROSS REFERENCE TO RELATED APPLICATIONS

This Application claims the benefit of U.S. provisional patent application Ser. No. 61/082,958 which is incorporated herein by reference.

### STATEMENT REGARDING FEDERALLY SPONSORED RESEARCH OR DEVELOPMENT

Not Applicable

### BACKGROUND

Certain biofuel processes, such as processes dedicated to the production of ethanol from biomass, are limited in their ability to take advantage flexibility of feed stock, limited by resonance time requirements, limited by lower than desired yields, and require high levels of externally supplied energy to run those processes. Processes having increased flexibility and efficiency are needed to address those needs. Conventional biofuel processes have not adequately met these needs.

Information relevant to attempts to address these limitations can be found in U.S. Pat. No. 5,711,817 to Titmas entitled “Method for the Continuous Conversion of Cellulosic Material to Sugar”; U.S. Pat. No. 7,504,245 to Kinley et al. entitled “Biomass Conversion to Alcohol Using Ultrasonic Energy”; United States Patent Office Pre-Grant Publication No. 2007/0141691 to Hirl entitled “Process for Producing Ethanol and for Energy Recovery”; and WIPO publication WO 2007/059777 to BIOACE I/S entitled “Biogas Plant and Process with Immobilized Bacteria.” However, none of these references has adequately solved the above described needs. For the forgoing reasons, there is a need for devices and methods that aid in addressing one or more of the above described needs.

### SUMMARY

Disclosed herein are embodiments of the present invention that address the needs described above by providing devices and methods that provide either increased efficiency or increased flexibility of feed stocks or both.

A process for the production of biofuel having features of the present invention comprises reducing a first biomass feedstock to granule or powder form; mixing the first biomass feedstock with water to create a mash; combining a second biomass feedstock with the first biomass feedstock wherein the second biomass feed stock is of a substantially different composition than the first biomass feedstock; exposing the mash to a shock wave that travels at least twice the speed of sound; exposing at least a component of the mash to a first microorganism causing fermentation; and distilling a fermentation product to produce a fuel. In an embodiment of the invention, the first biomass feedstock is a high starch content material. In a further embodiment of the invention, the second biomass feed stock contains a substantial fraction of sugar. In another embodiment of the invention, the first biomass feedstock is a high starch content material; the second biomass feed stock contains a substantial fraction of sugar; and the ratio of the first biomass feedstock to the second biomass feedstock is from about 2:1 to about 6:1. In a further embodiment of the invention, one of either the first biomass feedstock or the second biomass feedstock is a cellulosic material rich in five and six carbon organic molecules and in another related embodiment, a third biomass feedstock that is a cellulosic material rich in five and six carbon organic molecules is combined with the first biomass feedstock and the second biomass feedstock. In one embodiment of the invention, the reduction of the first biomass feed stock is accomplished by modified wet milling. In another embodiment of the invention, the mash contains a quantity of solid constituents and the solid constituents are disassociated to such an extent that 97% of any starch in the mash can by converted to sugar within a period of about 45 minutes or less. In still another embodiment of the invention, the shock wave is created by steam injection at three or more injection sites in series and the shock wave is delivered during liquefaction of starches and during hydrolysis. In an embodiment of the invention, a quantity of stillage from fermentation is exposed to a second microorganism in a heated, plug flow anaerobic biological reactor creating methane.

A process for the production of biofuel having features of the present invention comprises producing a biofuel by mixing a biomass feedstock with water to create a mash; hydrolyzing a constituent of the mash in the presence of a shock wave that travels at least twice the speed of sound; exposing at least a component of the mash to a micro-organism causing fermentation; distilling a fermentation product to produce a fuel; and digesting stillage remaining after fermentation anaerobically in a reactor vessel to produce a biogas and a solid waste.

A process for the production of ethanol having features of the present invention comprises providing a plant based feedstock comprised of at least 25 wt. % starch material; milling the plant based feedstock to an effective size to produce a powdered meal; converting the powdered meal into a mash by adding an effective amount of water to the powdered meal, which mash has a gel-like consistency; cooking the mash for an effective cooking time at an effective cooking temperature; hydrolyzing the cooked mash with use of a hydrolyzing agent for an effective amount of hydrolysis time and at an effective hydrolysis temperature and in the presence of a shock wave at least twice the speed of sound thereby liquefying a substantial portion of the cooked mash; adding an effective amount of yeast to the hydrolyzed cooked mash and subjecting it to fermentation thereby producing ethanol, carbon dioxide and stillage; distilling at least a component of the fermented hydrolyzed mash and separately collecting the ethanol, the carbon dioxide, and the stillage; and digesting the stillage anaerobically in a reactor vessel to produce a biogas and a solid waste. In an embodiment of the invention, an enzyme is added in one or more process steps including converting the meal to mash, cooking the mash, and hydrolyzing. That enzyme may be for example alpha amylase. In another embodiment of the invention the shock wave is generated by the injection of steam. In certain embodiment, the feedstock is a biomass comprised of a mixture of starch based materials, sugar based materials and cellulosic based materials, with the proviso that at least about 25 wt. % of the biomass feedstock be comprised of a starch based material. In some of those certain embodiments, the biomass starch based material is selected from the group consisting of corn, barley and wheat; the biomass sugar based material is selected from the group consisting of sugar beets, sugar cane, and molasses; and the biomass cellulosic based material is selected from the group consisting of straw, wood, paper waste, corn stover and switchgrass. Still other embodiments have a second shock wave of at least twice the speed of sound is provided during the anaerobic digestion of the wet solid residue material. In certain embodiments the fermentation is a continuous fermentation and in certain other embodiments the reaction vessel in which anaerobic digestion occurs is operated as a plug flow reactor with back mixing.

### BRIEF DESCRIPTION OF THE DRAWING

FIG. 1 shows a process step diagram of an embodiment of the invention that utilizes multiple feed stocks, a shock wave in the cooking and mashing step, continuous fermentation, and anaerobic digestion of stillage.

### DETAILED DESCRIPTION

Any one or more biomass materials can be used in the practice of the present invention. In an embodiment of the invention biomass materials are from renewable plant sources. Non-limiting examples of biomass materials that are used in the practice of the present invention include those that are starch-based, sugar-based, and cellulosic-based. By “-based” we mean that the material will contain more of the base component than any of the other carbohydrate components. For example, a starch-based biomass material will contain more starch than sugar or cellulosic material and a sugar-based biomass material will contain more of a sugar component than a starch or cellulosic component. In a further embodiment, at least about 50 wt. % of the total carbohydrate content of any starch-based, sugar-based, or cellulosic-based component is starch, sugar, or cellulose, respectively. Non-limiting examples of starch-based materials include corn, wheat, barley, potatoes, and millet. Preferred are those having a high starch content, such as corn, wheat and barley. Non-limiting examples of sugar-based biomass materials include sugar cane and sugar beets. In various embodiments of the invention, the sugar based biomass materials have a sugar content of at least about 15 wt. % inverted sugar, at least about 20 wt. % inverted sugar, at least about 25 wt. % inverted sugar or at least about 5 wt. % sucrose. Non-limiting examples of cellulosic materials include wood, woodchips, hemp, straw, cotton, switchgrass, corn stover, and bagasse. In an embodiment of the invention, the feedstock is comprised of a mixture of both the high starch and high sugar content materials. In two further embodiments, the ratio of high starch to high sugar material is from about 2:1 to about 6:1 or from about 5:2 to about 5:1. In a still further embodiment, cellulosic material rich in five and six carbon molecules are added to the feed stock.

In an embodiment of the invention, biofuel is produced by from one or more agricultural feedstocks such as sugar cane, bagasse, miscanthus, sugar beet, sorghum, grain sorghum, switchgrass, barley, hemp, kenaf, potatoes, sweet potatoes, cassava, sunflower, fruit, molasses, corn, stover, grain, wheat, straw, cotton, other biomass, as well as many types of cellulose waste and harvestings. In a further embodiment of the invention, biofuel is produced from two or more of the above agricultural feedstocks.

An embodiment of the invention combines the front end pretreatment of applying a sonic shock wave with the back end treatment of biosolids in an anaerobic digester. Such a design allows for greater flexibility in feedstock mix to the plant, since traditional constraints around the liquefaction/fermenter and stillage handling systems are substantially removed.

One embodiment of the present invention is a multi-step process for producing ethanol from the biomass feedstock and optionally a biogas, such as methane, which can be sold to a third party or used as energy for one or more of the process steps. In a first step, the feedstock is prepared for processing by first milling it to an effective particle size, thereby resulting in a powdered feedstock. In certain embodiments, the particle size is a substantially uniform particle size. The milling can be dry milling, wet milling, or “modified” wet milling.

Conventional dry milling ethanol plants typically convert the biomass, usually corn, into ethanol, carbon dioxide and a solid residue conventionally referred to as distiller's grains with solubles when the feedstock is one such as corn. If sold as wet animal feed, distiller's wet grains with solubles are referred to as DWGS. If dried for animal feed, distiller's grains with solubles are referred to as DDGS. This co-product provides a secondary revenue stream that offsets a portion of the overall ethanol process. In an embodiment of the present invention wherein the biomass feedstock is blend of starch, sugar, and cellulosic based materials, conventional distiller's wet grains will not be produced. Thus, the solid material will be referred to herein as wet solid residue material or stillage. In an embodiment of the invention, this wet solid residue material is subjected to anaerobic digestion to produce a biogas, such as methane, that can be used as fuel for one or more of the steps to the instant process. The ability to anaerobically digest the solid residue material while wet has a significant energy saving for the instant process. It also removes process constraints around the evaporators and driers traditionally associated with starch-based feedstocks. The anaerobic digestion step will be discussed in more detail below.

In an embodiment of the invention, wet milling of the biomass feedstock is used. Wet milling can be utilized to convert a biomass feedstock, such as corn grain, into several different co-products, such as germ (for oil extraction), gluten feed (high fiber animal feed), gluten meal (high protein animal feed), and starch-based products such as ethanol, high fructose corn syrup, or food and industrial starch.

In a further embodiment of the invention, modified wet milling is used. Modified wet milling is a variation of wet milling which eliminates much of the capital investment required for complete corn fermentation. In modified wet milling, a shorter steeping cycle is used, which allows only for germ separation. There is no separate gluten or fiber recovery section, as in conventional wet milling. The separated germ slurry, which contains most of the oil found in corn, is dewatered and dried for co-product sales. Modified wet milling does not produce a clean starch stream, as gluten and fiber components are carried along with starch into the hydrolysis and fermentation steps. A final co-product is produced with characteristics similar to DDGS generated from dry milling, except much of the oil is removed. For purposes of the this embodiment wherein the biomass feedstock can be a blend of starch, sugar and cellulosic based material, the resulting solid residue will be of a lower quality than conventional DDGS and possibly not fit as animal feed.

In an embodiment of the invention in which dry milling is used, at least a portion of the powdered feedstock is mixed with an effective amount of water to form a slurry, sometimes called a mash, having a gel-like consistency. The mash is heated to a temperature of about 40° to about 120° C, and in some embodiments is heated to temperature of about 50° to about 90° C. In an embodiment of the invention, the mash is cooked for an effective amount of time. An effective cooking time will be that minimum amount of time needed to gelatinize (solubilize) the starch in the ground meal. In certain embodiments the cooking of the mash is done under pressure. Embodiments contain cooking times from about 5 to about 30 minutes, with certain embodiments having cooking times from about 5 to about 20 minutes. An enzyme, such as alpha amylase, can be added prior to liquefaction but the amounts of this enzyme and any other enzyme will depend on how effective the mashing and liquefaction under shock wave technology are and of the composition of the feedstock at the front end of the process. In an embodiment of the invention, the cooking process is performed in a jet cooker. Jet cooking refers to a cooking process performed at elevated temperatures and pressures, although the specific temperatures and pressures can vary widely. The jet cooking occurs at a temperature of about 120° to 150° C. and a pressure of about 8.4 to 10.5 kg/cm² (about 120 to 150 lbs/in²), although the temperature can be as low as about 104° to 107° C. when pressures of about 8.4 kg/cm² (about 120 lbs/in²) are used. In an alternate embodiment, a non-jet cooking process is used, in which the temperature is less than the boiling point, such as about 90° to 95° C. (about 194° to 203° F.) or lower, down to about 80° C. (176° F.). In this embodiment, at these lower temperatures, ambient pressure would be used.

The cooked mash is then subjected to liquefaction by hydrolysis for an effective amount of time. The hydrolysis can be by any conventional method including enzyme hydrolysis as well as acid hydrolysis. By effective amount of time, we mean from about 1.5 to 4 hours, with individual embodiments taking from about 2 to 3 hours. The hydrolysis is used to convert the starch and/or cellulose and/or maltodextrins and/or maltose to fermentable sugar (saccharification). The hydrolysis can be by any conventional method including enzyme hydrolysis as well as acid hydrolysis.

In an embodiment of the present invention, liquefaction is enhanced during hydrolysis by subjecting the cooked mash to a shock wave. That is, for at least that amount of time needed to reach a conversion of 97% starch to glucose in a reduced time of about 45 minutes. In certain embodiments of the invention, the shock wave treatment is sufficient to reach a conversion of 97% starch to glucose in 30 minutes or less. In an embodiment of the invention, the liquefing of up to 99% of starch and protein of the solids in the mash to a liquid is achieved. Conventional hydrolysis methods, without the use of shock waves, typically require residence times of about 1.5 to 4 hours to achieve <90% starch conversion. By controlling the amount and intensity of the sonic shock wave applied here, variations in feedstock selection can be accommodated. In addition, the sonic shock wave increases the amount of starch conversion to glucose, hence resulting in higher ethanol production.

In an embodiment of the invention, the shock wave is produced by injecting steam into the hydrolysis vessel at velocities at least two times the speed of sound. In a further embodiment, the shock wave is produced by injecting steam into the hydrolysis vessel at velocities at least about 3 times the speed of sound. The introduction of the shock wave during alcohol production enhances the restructuring, disaggregation, and disassociation of starch granules from other grain components, such as protein and fiber, which may inhibit the conversion of starch to glucose and ethanol. The shock wave is able to loosen, shake off and/or strip away starch granules from protein bodies, protein matrices, and fiber (fine or coarse), as well as disassociate tightly packed granules and tightly packed amyloplasts which contain starch granules. In certain embodiments of the invention the shockwave technology accomplishes almost total conversion from starch to glucose. This high conversion reduces liquefaction and hydrolysis times. It will be noted that over-processing of the components, e.g., over-processing of starch prior to fermentation, is not desirable. Specifically, if the shock wave is too aggressive in terms of intensity, frequency and/or duration, it may be possible to cause some damage to the components being treated. For example, care must be taken not to degrade desirable enzymes. Additionally, care must also be taken to not shear the starch so much that later fermentation is inhibited or stalled. In an embodiment of the invention, the sonic devices are 3 to 6 devices in a series, each delivering a short sonic burst of less than 10 seconds at a predetermined angle and rate. The optimum design of the angle and rate for the sonic devices is dependent on the material being treated and will be determined via pilot testing to account for specific properties of the solid materials being treated. For example, the angle and rate used for the sonic device design to pre-treat the feed stock materials in the hydrolysis stage would not be the same as that used to pre-treat the stillage prior to anaerobic digestion. Certain embodiments of the invention utilizing shock waves have one or more of the following features: the liquefaction process is accomplished in less than 45 minutes, and in certain instances less than 30 minutes; heat is added via the injected steam, further reducing the energy requirements; and the starch is fully treated, aiding fermentation by reducing fermentation time and increasing starch to ethanol conversion by as much as 15% compared to conventional means.

An effective amount of suitable enzymes and yeast are added to the resulting liquefied product stream from hydrolysis, which is then sent to a fermentation step resulting in a product stream comprised of ethanol, carbon dioxide, and wet solid residue. In an embodiment of the invention, the fermentation step is a continuous fermentation step. The fermentation product stream containing alcohol is conducted to a distillation zone. In an embodiment of the invention the distillation is run in a continuous mode. The ethanol is collected and passed to a drying zone wherein substantially all of the water content is removed. The wet solid residue is not dried. It is sent wet to an anaerobic digestion zone in the presence of anaerobic microbes and digested to produce a biogas, preferably methane, and a liquid slurry waste stream. The conversion of organic molecules to methane by anaerobic microbes must occur in an environment void of oxygen. The rate of conversion will be proportional to the temperature of the environment and the number of microbes in the system. In an embodiment of the invention, the digestion is performed by use of a heated plug flow anaerobic biological reactor. In certain embodiments, the plug flow reactor has back mixing and in still other embodiments, a continuous stirred tank reactor (CSTR) digester is used. In an embodiment of the invention, hydraulic retention times for digestion system are at least about two days. In another embodiment of the invention, hydraulic retention times for digestion system are from about 2 days to about 3 days. Biogas is collected in the reactor headspace. In embodiments utilizing a CSTR, after 14 to 20 days in the reactor the resulting effluent slurry is passed to a dewatering zone wherein a substantial amount of water is removed, such as by use of a screw press. In certain embodiments having a plug flow reactor with back mixing, the processing time will reduce to 1 to 4 days, especially if a sonic shock wave is used to pre-treat the solids prior to sending to the digester. The resulting solids are collected and can be used for such things as dairy bedding or an organic soil amendment. The resulting liquid can be recycled to the slurry tank or used as a liquid component of a fertilizer. The biogas can be used as a fuel for a generator to convert the same to electricity and hot water. The digester can be heated using a portion of heat generated by either a boiler or from the waste heat produced by an electrical generator. The electricity can be used to run the ethanol facility. The fuel could also be sent to a boiler to raise steam for use in the process and to supply the sonic devices.

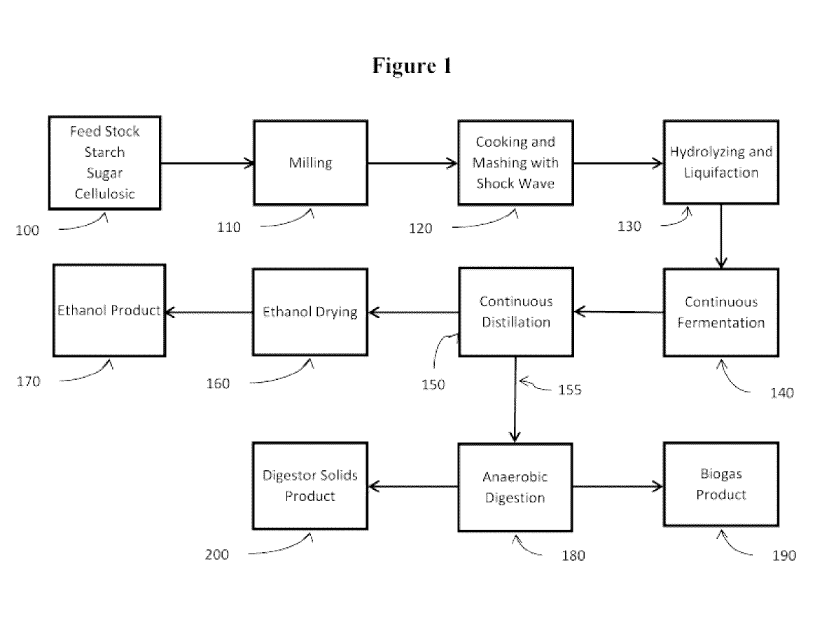

Now referring to FIG. 1 of the drawings, which is a process step diagram of an embodiment of the invention, feed stock 100 is milled in a milling step 110 as may be necessary to make the feedstock suitable for cooking and mashing. Cooking and mashing with a shock wave 120 is performed on the biomass followed by the hydrolyzing and liquefaction step 130. Sugars from the step 130 are fermented in a continuous fermentation step 140 producing an aqueous ethanol product. The ethanol product is concentrated in a continuous distillation 150 and ultimately dried in an ethanol drying step 160 to produce an ethanol product 170 suitable for use as a fuel. Stillage 155 from the ethanol process is separated from the process and subjected to anaerobic digestion step 180 in which a biogas product 190 and a digester solids product 200 are produced.

Further embodiments of the invention include the making of a wide range of biofuels other than ethanol utilizing the teachings herein, including the making of biodiesel, biobutanol, and other fuels.

Any and all reference to patents, documents and other writings contained herein shall not be construed as an admission as to their status with respect to being or not being prior art. It is understood that the array of features and embodiments taught herein may be combined and rearranged in a large number of additional combinations not directly disclosed, as will be apparent to one having skill in the art.

There are, of course, other alternate embodiments which are obvious from the foregoing descriptions of the invention, which are intended to be included within the scope of the invention, as defined by the following claims.

We claim:

1. A process of producing a biofuel comprising:
a. reducing a first biomass feedstock to granule or powder form;
b. mixing the first biomass feedstock with water to create a mash;
c. combining a second biomass feedstock with the first biomass feedstock wherein the second biomass feed stock is of a substantially different composition than the first biomass feedstock;
d. exposing the mash to a shock wave that travels at least twice the speed of sound;
e. exposing at least a component of the mash to a first microorganism causing fermentation; and
f distilling a fermentation product to produce a fuel.

2. The process of claim 1 wherein the first biomass feedstock is a high starch content material.

3. The process of claim 1 wherein the second biomass feed stock contains a substantial fraction of sugar.

4. The process of claim 1 wherein:
a. the first biomass feedstock is a high starch content material;
b. the second biomass feed stock contains a substantial fraction of sugar; and
c. the ratio of the first biomass feedstock to the second biomass feedstock is from about 2:1 to about 6:1.

5. The process of claim 1 wherein one of either the first biomass feedstock or the second biomass feedstock is a cellulosic material rich in five and six carbon organic molecules.

6. The process of claim 1 wherein a third biomass feedstock that is a cellulosic material rich in five and six carbon organic molecules is combined with the first biomass feedstock and the second biomass feedstock.

7. The process of claim 1 wherein the reduction of the first biomass feed stock is accomplished by modified wet milling.

8. The process of claim 1 wherein the mash contains a quantity of solid constituents and the solid constituents are disassociated to such an extent that 97% of any starch in the mash can by converted to sugar within a period of about 45 minutes or less.

9. The process of claim 1 wherein:
a. the shock wave is created by steam injection at three or more injection sites in series and
b. the shock wave is delivered during liquefaction of starches and during hydrolysis.

10. The process of claim 1 wherein a quantity of stillage from fermentation is exposed to a second microorganism in a heated, plug flow anaerobic biological reactor creating methane.

11. A process of producing a biofuel comprising:
a. Mixing a biomass feedstock with water to create a mash;
b. hydrolyzing a constituent of the mash in the presence of a shock wave that travels at least twice the speed of sound;
c. exposing at least a component of the mash to a micro-organism causing fermentation;
d. distilling a fermentation product to produce a fuel; and
e. digesting a stillage component remaining after fermentation anaerobically in a reactor vessel to produce a biogas and a solid waste.

12. A process wherein ethanol is produced from a renewable plant based feedstock, which process comprises:
a. providing a plant based feedstock comprised of at least 25 wt. % starch material;
b. milling the plant based feedstock to an effective size to produce a powdered meal;
c. converting the powdered meal into a mash by adding an effective amount of water to the powdered meal, which mash has a gel-like consistency;
d. cooking the mash for an effective cooking time at an effective cooking temperature;
e. hydrolyzing the cooked mash with use of a hydrolyzing agent for an effective amount of hydrolysis time and at an effective hydrolysis temperature and in the presence of a shock wave at least twice the speed of sound thereby liquefing a substantial portion of the cooked mash;
f. adding an effective amount of yeast to the hydrolyzed cooked mash and subjecting it to fermentation, thereby producing ethanol, carbon dioxide and stillage;
g. distilling at least a component of the fermented hydrolyzed mash and separately collecting the ethanol, the carbon dioxide, and the stillage; and
h. digesting the stillage anaerobically in a reactor vessel to produce a biogas and a solid waste.

13. The process of claim 12 wherein an enzyme is added in one or more of step c., step d., and step e.

14. The process of claim 13 wherein the enzyme is alpha amylase.

15. The process of claim 12 wherein the shock wave is generated by the injection of steam.

16. The process of claim 12 wherein the feedstock is a biomass comprised of a mixture of starch based materials, sugar based materials and cellulosic based materials, with the proviso that at least about 25 wt. % of the biomass feedstock be comprised of a starch based material.

17. The process of claim 16 wherein the biomass starch based material is selected from the group consisting of corn, barley and wheat; the biomass sugar based material is selected from the group consisting of sugar beets, sugar cane, and molasses; and the biomass cellulosic based material is selected from the group consisting of straw, wood, paper waste, corn stover and switchgrass.

18. The process of claim 12 wherein a second shock wave of at least twice the speed of sound is provided in step h. during the anaerobic digestion of the wet solid residue material.

19. The process of claim 12 wherein the fermentation is a continuous fermentation.

20. The process of claim 12 wherein the reaction vessel in which anaerobic digestion occurs is operated as a plug flow reactor with back mixing.
